(19) United States
(12) Patent Application Publication
(10) Pub. No.: US 2014/0073053 A1
(43) Pub. Date: Mar. 13, 2014

(54) METHODS FOR TRANSFECTING CELLS WITH NUCLEIC ACIDS

(71) Applicant: Massachusetts Institute of Technology, Cambridge, MA (US)

(72) Inventors: Mehmet Fatih Yanik, Watertown, MA (US); Matthew Angel, Cambridge, MA (US)

(73) Assignee: Massachusetts Institute of Technology, Cambridge, MA (US)

(21) Appl. No.: 14/020,356

(22) Filed: Sep. 6, 2013

Related U.S. Application Data
(63) Continuation of application No. PCT/US2012/028146, filed on Mar. 7, 2012.
(60) Provisional application No. 61/450,116, filed on Mar. 7, 2011.

(51) Int. Cl. C12N 15/85

(52) U.S. Cl. CPC C12N 15/85
USPC 435/455; 435/377; 435/406

(57) ABSTRACT

The present disclosure provides culture media and methods of using culture media for efficient transfection of a target cell with nucleic acid molecules. The media is capable of supporting cells in culture that are differentiating, transdifferentiating, and/or dedifferentiating.

### CROSS REFERENCE TO RELATED APPLICATIONS

This application is a continuation of International Application Number PCT/US2012/028146, filed Mar. 7, 2012, which claims priority to U.S. Provisional Patent Application Ser. No. 61/450,116, filed Mar. 7, 2011, the entirety of each of which is incorporated herein by reference.

### BACKGROUND

RNA transfection is a powerful method for expressing high levels of proteins both in vitro and in vivo that avoids the risk of mutation associated with DNA-based methods. However, long in vitro-transcribed RNA molecules induce a potent innate immune response that causes cell death. It has been demonstrated that suppressing the innate immune response of target cells to transfection with exogenous RNA (herein used synonymously with “in vitro-transcribed RNA” (ivT-RNA)) facilitates frequent repeated transfections with exogenous RNA encoding various proteins of interest, including reprogramming proteins (see US Patent Appl. Pub. No. US 2010/0273220, Angel & Yanik (2010) PLoS One 5:1-7)). Proteins involved in the innate immune response include, for example, TP53, TLR3, TLR7, RARRES3, IFNA1, IFNA2, IFNA4, IFNA5, IFNA6, IFNA7, IFNA8, IFNA10, IFNA13, IFNA14, IFNA16, IFNA17, IFNA21, IFNK, IFNB1, IL6, TICAM1, TICAM2, MAVS, STAT1, STAT2, EIF2AK2, IRF3, TBK1, CDKN1A, CDKN2A, RNASEL, IFNAR1, IFNAR2, OAS1, OAS2, OAS3, OASL, RB1, ISG15, ISG20, IFIT1, IFIT2, IFIT3, and IFIT5, or a biologically-active fragments, analogs or variants thereof.

### SUMMARY

Methods for dedifferentiating cells are important to the fields of drug-discovery and cell-replacement therapy (also known as “regenerative medicine”). Pharmaceutical companies screen large libraries of compounds using cell-based assays to identify novel therapeutics. However, there is currently no method for generating the large quantities of disease-specific and tissue-specific cells needed for these screens. As a result, most high-throughput screens are conducted using immortalized cells that can not accurately recapitulate the disease state in vitro because of the phenotypic abnormalities caused by the immortalization process. In addition to the risk of mutation associated with other methods for dedifferentiating cells, existing methods for dedifferentiating cells are inefficient. Thus, there is a need for increasing the efficiency with which cells can be dedifferentiated.

Various media are used for the culture of cells in vitro. Culture media are designed to provide cells with the nutrients required to maintain their viability, and in the case of proliferating cells, to support their growth. Specialized culture media have been developed to support the growth of certain specific cell types, including pluripotent stem cells, and other culture media are useful for dedifferentiating somatic cells (such as fibroblasts) into a pluripotent stem cell state using viruses or other DNA-based methods. However, these media cannot be used for certain applications, such as to efficiently dedifferentiate cells to a pluripotent stem cell state using exogenous/ivT-RNA encoding reprogramming proteins. Such applications require that the culture medium support the growth of somatic cells as well as the dedifferentiated pluripotent stem cells, while supporting efficient transfection with ivT-RNA encoding reprogramming proteins without stimulating the differentiation of dedifferentiated cells or partially-dedifferentiated cells. Thus, there is a great need for culture media that meet these criteria and support high efficiency transfection with exogenous RNA, particularly long ivT-RNA molecules.

Described herein are methods and compositions for transfection of a target cell with nucleic acids molecules. In certain embodiments, media are provided for transfecting a target cell with a ribonucleic acid molecule. In certain embodiments, methods for transfecting a target cell with a ribonucleic acid molecule are provided. The methods comprise suppressing the innate immune response in the target cell, and introducing the ribonucleic acid molecule into the target cell, wherein the target cell is cultured in a medium described herein.

### BRIEF DESCRIPTION OF THE DRAWINGS

FIG. 1 provides a bar graph comparing the upregulation of innate immune-related genes in cells transfected with modified ivT-RNA, and cultured either in the presence or absence of the immunosuppressant protein B18R. mRNA was extracted from the transfected cells, and gene expression was measured by quantitative RT-PCR. Gapdh was used as a loading control. Error bars indicate the standard error of replicate samples (n=2).

FIG. 2 depicts MRC-5 fibroblasts transfected every day for five days with 1.2 ug/well of modified mRNA encoding Oct4, Sox2, Klf4, c-Myc (T58A), Lin28, and destabilized nuclear GFP, and cultured either in the presence or absence of the immunosuppressant protein B18R.

FIG. 3 provides a graph illustrating the change in cell density of the cells depicted in FIG. 2 over time. Samples of cells were trypsinized and counted at the indicated times. Error bars indicate the standard error of replicate samples (n=4).

FIG. 4 depicts the expression of GFP in cells repeatedly transfected with modified mRNA. The cells depicted in FIG. 2 were imaged for GFP fluorescence. Identical camera settings and exposure times were used to capture each image. Two random fields are shown for each sample.

FIG. 5 depicts representative images of transfected cells on day 5 showing GFP fluorescence only in cells cultured with B18R.

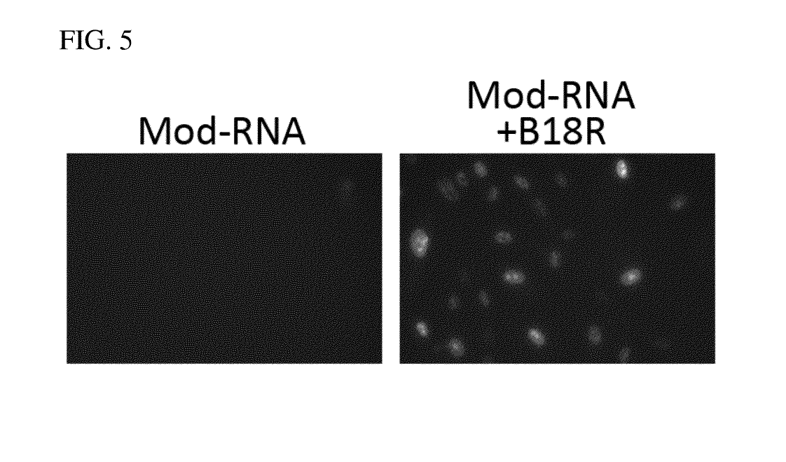

FIG. 6 depicts protein translation from modified mRNA containing the modified nucleotides pseudouridine and 5-methylcytidine. MRC-5 fibroblasts were transfected with Oct4-encoding mRNA containing complete substitution with pseudouridine (Ψ) and/or 5-methylcytidine (5mC) and either the Cap 0 or Cap 1 5′ cap. Cells were fixed and stained 12 hours after transfection. Identical camera settings and exposure times were used to capture each image. Two random fields are shown for each sample.

FIG. 7 provides a bar graph comparing the relative protein translation from RNA containing various combinations of the modified nucleotides pseudouridine and 5-methylcytidine. The images in FIG. 6 were analyzed by first determining a background threshold by taking the maximum pixel intensity outside a cell nucleus, and subtracting that value from all of the pixels, and then calculating the mean pixel intensity. The same threshold was used for all of the images. Error bars indicate the standard error of intensity from the two random fields.

FIG. 8 depicts fibroblasts transfected with ivT RNA encoding a plurality of reprogramming proteins, and cultured in a medium containing the immunosuppressant B18R and a high concentration (2 ng/mL) of TGF-beta. Arrows indicate areas of cells that began to dedifferentiate, but then ceased dedifferentiating due to the high concentration of TGF-beta present in the culture medium.

FIG. 9 depicts fibroblasts transfected as in FIG. 8, and cultured in a medium containing the immunosuppressant B18R, not containing TGF-beta, and not containing a surfactant.

FIG. 10 depicts GFP fluorescence in fibroblasts transfected as in FIG. 8, and cultured in medium containing both the immunosuppressant B18R and the surfactant Pluronic F-68, and not containing TGF-beta.

FIG. 11 depicts BJ (human foreskin) fibroblasts transfected and cultured as in FIG. 10. Arrows indicate cells undergoing dedifferentiation.

### DETAILED DESCRIPTION

As used herein, “transfection” refers to any method of delivering a nucleic acid to a cell, including pre-complexing the nucleic acid with a lipid-based or peptide-based or polymer-based material and then delivering the pre-complexed nucleic acid to the cell.

As used herein, “surfactant” refers to any molecule with amphiphilic properties or any molecule that lowers the surface tension of a liquid, the interfacial tension between two liquids, or the interfacial tension between a liquid and a solid.

As used herein, “culture medium” refers to any solution capable of sustaining the growth of the targeted cells either in vitro or in vivo, or any solution with which targeted cells or exogenous nucleic acids are mixed before being applied to cells in vitro or to a patient in vivo.

As used herein, “stem cell” refers to any cell capable of differentiating into another cell type, either in vitro or in vivo.

As used herein, “somatic cell” refers to any cell that is not a stem cell.

As used herein, media that are “substantially free of TGF-beta” refers to media that are devoid of TGF-beta, or have not had TGF-beta added to said media, or contain only trace amounts of TGF-beta such that TGF-beta activity does not adversely affect the ability of somatic cells to dedifferentiate.

Methods for dedifferentiating human fibroblasts to a pluripotent stem cell state have been reported (see, e.g., US Patent Appl. Pub. No. US 2010/0273220 by Angel & Yanik; Warren et al. (2010)). These methods include repeated delivery of RNA (transfection of targeted cells) encoding reprogramming proteins using a culture medium containing one or more agents that suppress the innate immune response.

It is discovered herein that transfection with exogenous RNA using any method of transfection may be efficiently performed when the targeted cells are contacted with or cultured in a medium that is substantially free of TGF-beta.

In certain embodiments media are provided for transfecting a target cell with a ribonucleic acid molecule. In certain embodiments, a medium is provided comprising DMEM/F12, L-alanyl-L-glutamine, insulin, transferring, selenous acid, cholesterol, cod liver oil fatty acids (methyl esters), polyoxyethylenesorbitan monooleate, D-alpha-tocopherol acetate, L-ascorbic acid 2-phosphate sesquimagnesium salt hydrate, and bFGF, wherein the medium is substantially free of TGF-beta.

In certain embodiments, a medium is provided consisting essentially of DMEM/F12, L-alanyl-L-glutamine, insulin, transferring, selenous acid, cholesterol, cod liver oil fatty acids (methyl esters), polyoxyethylenesorbitan monooleate, D-alpha-tocopherol acetate, L-ascorbic acid 2-phosphate sesquimagnesium salt hydrate, and bFGF, wherein the medium is substantially free of TGF-beta.

In certain embodiments, the medium further comprises human serum albumin.

In certain embodiments, the medium further comprises a surfactant. In certain aspects, the surfactant is a non-ionic surfactant.

Non-ionic surfactants include, but are not limited to, compounds according to the following formula I:

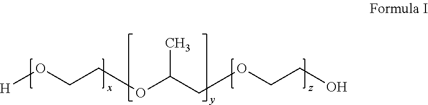

wherein x, y, and z are integers.

Examples of nonionic surfactants include, but are not limited to, PLURONIC F-68 (also known polyoxyethylene-polyoxypropylene block copolymer; C3H60.C2H40)x; CAS 9003-11-06; Pub Chem Substance ID: 24898182; SIGMA catalog number P5556) and PLURONIC F-127 (SIGMA catalog number P2443).

In certain aspects, the amount of surfactant in the medium is from about 0.01% to about 1%. In one aspect, the amount of the surfactant is about 0.1%

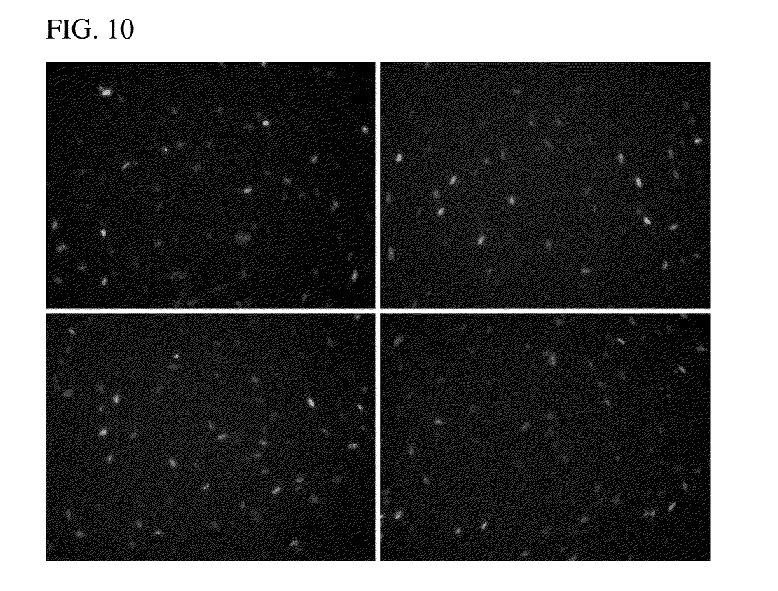

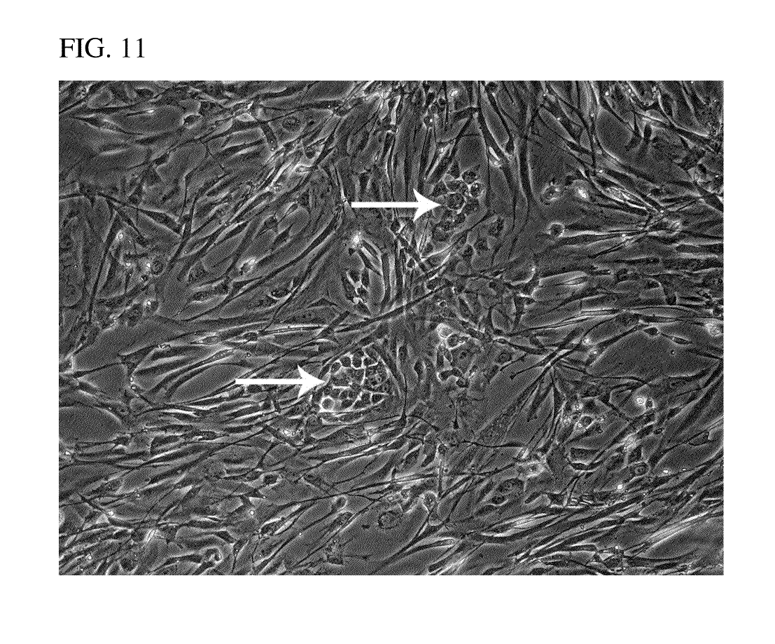

Surfactants have been used in large-scale cell culture to increase cell viability by reducing hydrodynamic stress. However, in small-scale cell culture surfactants are not typically used because of the low hydrodynamic forces generated in these systems. Use of a medium described herein containing a surfactant in an amount from about 0.01% to about 1%, can increase the efficiency of dedifferentiation of targeted cells repeatedly transfected with exogenous RNA encoding reprogramming proteins. See FIG. 10 and FIG. 11, and Example 5.

In certain embodiments, one or more immunosuppressive agents (immunosuppressants) are included in the medium.

In certain embodiments, the immunosuppressive agent is a protein. In certain embodiments, the immunosuppressive agent is B18R.

In certain embodiments, the immunosuppressive agent is a small molecule.

In certain embodiments, the small molecule is a steroid, including, but not limited to, dexamethasone.

The media described herein support growth of a somatic cell, growth of a stem cell, and dedifferentiation of a cell transfected with a ribonucleic acid molecule.

Methods for transfecting a target cell with a ribonucleic acid molecule are also provided. In certain embodiments, the methods comprise suppressing the innate immune response in the target cell; and introducing the ribonucleic acid molecule into the target cell, wherein the target cell is cultured in a medium as described herein.

In certain embodiments, introduction of the ribonucleic acid molecule produces a phenotypic change in the target cell. The phenotypic change in the target cell may include differentiation, transdifferentiation, and/or dedifferentiation. In certain embodiments the phenotypic change is dedifferentiation of the somatic cell to a multi- or pluripotent stem cell.

In certain embodiments, the target cell is a somatic cell. In certain embodiments, the cell is a somatic cell and the protein(s) of interest are reprogramming proteins that facilitate either differentiation of the target cell into a desired phenotype, or transdifferentiation, or alternatively the encoded proteins facilitate dedifferentiation of the somatic cell into a multi- or pluripotent stem cell. It has been discovered herein that culture media substantially free of TGF-beta facilitates dedifferentiation of cells.

In certain embodiments, cell that have been produced by the methods described herein are provided. The cells may be used, for example, as therapeutic agents or in applications for the screening of therapeutic compounds.

In certain embodiments the efficiency of transfection with exogenous ribonucleic acid molecules (RNA) is improved by contacting the target cells with a medium that contains a surfactant, either before or simultaneously with contacting the cells with the exogenous RNA (ivT-RNA) encoding one or more proteins of interest.

Media described herein are useful, for example, for improving dedifferentiation methods, such as the methods disclosed in US Patent Appl. Pub. No. US 2010/0273220, incorporated herein by reference in its entirety. Methods using the media described herein can be used to generate the cells needed for high-throughput screening. To accomplish this, cells from a patient are first dedifferentiated by contacting them with culture medium comprising a surfactant and preferably an immunosuppressant agent, simultaneously or before transfection with ivT RNA. The dedifferentiated cells are then expanded in number in culture, before being induced to differentiate into tissue-specific cell types using established methods (Cooper et al. (2010)). Because the cells are not immortalized, they more accurately recapitulate the disease state in vitro and, importantly, they have not been transformed with any potentially dangerous viruses or other exogenous DNA molecules.

Many diseases and injuries are characterized by the loss of defined populations of cells can be treated by transplantation, in which tissue from an HLA-matched donor is removed from the donor and then implanted into the recipient. However, this procedure carries great risks for both the donor and recipient, including risks associated with surgery and the removal of functional tissue for the donor, and the risks associated with surgery and immune rejection for the recipient. In addition, there is a constant shortage of donors for most tissue types. Methods for differentiation, transdifferentiation, and/or dedifferentiation of target cells, including those disclosed in US Patent Appl. Pub. No. US 2010/0273220, are improved by using the media described herein. Such improved methods can be used to generate autologous cells and tissues for cell-replacement therapies. To accomplish this, cells from a patient are first differentiated, transdifferentiated, and/or dedifferentiated as herein to obtain cells of the desired cell type required by the patient. These cells are then implanted into the patient, either alone or in combination with a scaffold or other apparatus, where they restore the function of the lost tissue. Ongoing cultures can be maintained for further use.

The media described herein are also useful in in vitro and in vivo applications including, but not limited to, dedifferentiation, differentiation, transdifferentiation, neural regeneration, and the over-expression of therapeutic proteins. Methods for delivering nucleic acids to target cells in vivo suffer from many of the same problems associated with methods for delivering nucleic acids to cells in vitro, including the problem of low transfection efficiency.

The efficacy of transfecting cells with exogenous RNA (or other nucleic acids) encoding any protein of interest is increased by the compositions and methods described herein. The following examples describe some exemplary modes of making and using the media certain compositions that are described herein. It should be understood that these examples are for illustrative purposes only and are not meant to limit the scope of the compositions and methods described herein.

### EXAMPLES

### Example 1

### Materials and Methods

Cell Culture. Primary human fetal lung fibroblasts (MRC-5), and newborn skin fibroblasts (BJ) were obtained from the ATCC and were cultured in DMEM+10% FBS. The immunosuppressant B18R (eBioscience) was used at a concentration of 200 ng/mL.

In Vitro-Transcription. dsDNA templates were prepared previously described, and were cloned into the pCR-Blunt II-TOPO vector using the Zero Blunt TOPO PCR Cloning Kit (Invitrogen). Plasmids were linearized by digestion with EcoRI (NEB), and were subjected to 10 cycles of PCR using a high-fidelity polymerase (KAPA HiFi, Kapa Biosystems). The amplified template was gel purified before in vitro transcription. Capped, poly(A)+RNA was synthesized using the mSCRIPT mRNA Production System (EPICENTRE). Where indicated, pseudouridine-triphosphate and 5-methylcytidine-triphosphate (TRILINK) were substituted for UTP and CTP, respectively. To generate mRNA containing the Cap 0 structure, the 2′-O-methyltransferase was omitted from the capping reaction. Transcripts were analyzed both before and after poly(A) tailing by denaturing formaldehyde-agarose gel electrophoresis. Primers used for assembly of in vitro-transcription templates have been previously disclosed (Angel & Yanik (2010)).

mRNA Transfection. Lipid-mediated transfections (Lipofectamine RNAiMAX, Invitrogen) were performed according to the manufacturer's instructions. The culture medium was replaced 4 hours after each transfection.

Quantitative RT-PCR. RNA was extracted using RNEASY kits (QIAGEN). TAQMAN Gene Expression Assays (APPLIED BIOSYSTEMS) were used in one-step RT-PCR reactions (ISCRIPT ONE-STE RT-PCR Kit, BIO-RAD) consisting of a 50° C., 10 min reverse transcription step, followed by an initial denaturation step of 95° C. for 5 min, and 45 cycles of 95° C. for 15 sec and 55° C. for 30 sec.

Immunocytochemistry. Cells were rinsed in TBST and fixed for 10 minutes in 4% paraformaldehyde. Cells were then permeabilized for 10 minutes in 0.1% TRITON X-100, blocked for 30 minutes in 1% casein, and incubated with appropriate antibodies (Angel & Yanik (2010)).

### Example 2

### Modified RNA is Immunogenic

In vitro-transcribed (ivT) mRNA is a powerful tool for expressing defined proteins both in vitro and in vivo, and avoids the mutation risks associated with DNA-based vectors. Although ivT mRNA is quickly translated by cells into high levels of functional protein, cells respond to repeated transfection with ivT mRNA as they do to infection with RNA virus: by halting cell growth, upregulating receptors for exogenous RNA, and secreting inflammatory cytokines, which hypersensitize nearby cells. It has recently been demonstrated that inhibition of two components of the innate immune system, type I-interferon signaling and activation of protein kinase R (PKR), rescues cells from the cell death caused by frequent transfection with ivT mRNA (Angel & Yanik (2010)). It has further been shown that repeated ivT mRNA transfection enables sustained expression of functional proteins, and this technique can be used to express reprogramming factors in primary human fibroblasts.

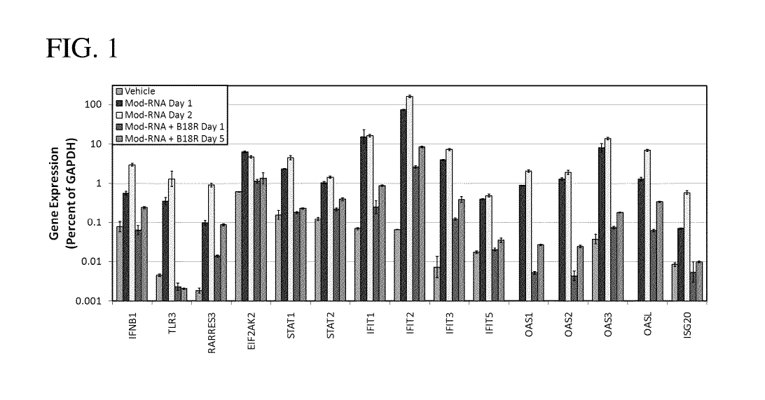

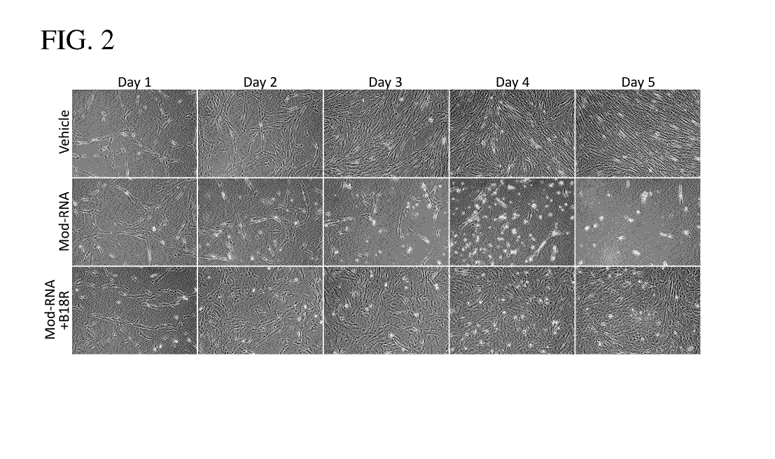

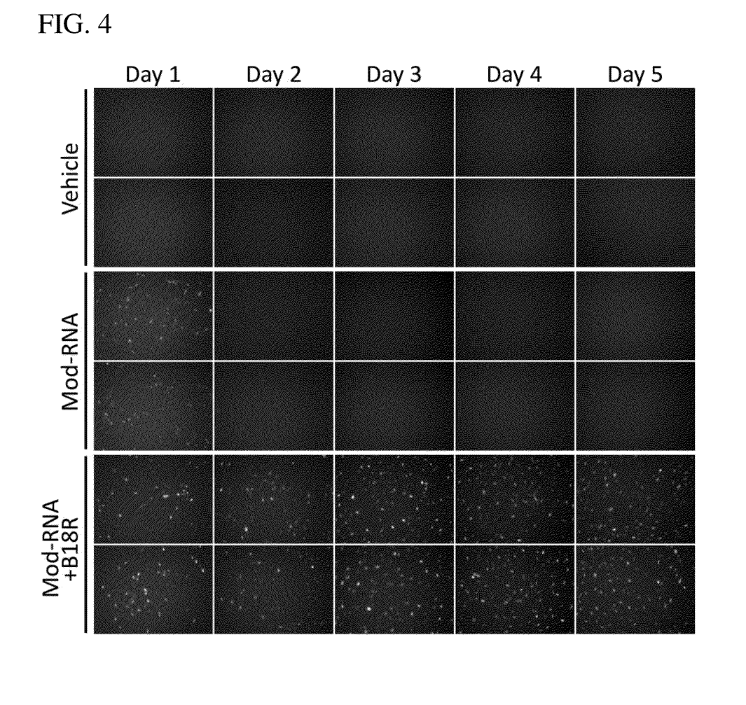

The incorporation of certain modified nucleotides has been suggested as a method for reducing the immunogenicity of ivT mRNA (Warren et al. (2010); Kormann et al. (2011)). However, in the present experiments discussed herein, it has been found that single transfection with modified mRNA triggers a potent innate immune response in human fibroblasts characterized by >100-fold upregulation of several interferon-stimulated genes including IFIT1,2, and 3 and >50-fold upregulation of the receptors of exogenous RNA, TLR3 and RIG-I (FIG. 1). Subsequent daily transfections resulted in further upregulation of immune-related genes (FIG. 1), elimination of encoded-protein expression (FIGS. 4,5), and massive cell death (FIGS. 2,3). Supplementation of the culture medium with a potent and specific inhibitor of type I-interferon signaling (the protein B18R) resulted in reduced upregulation of immune-related genes (FIG. 1), sustained, high-level expression of the encoded protein (FIGS. 4,5), and proliferation at a rate indistinguishable from the mock-transfected control (FIGS. 2,3). These results demonstrate that transfection with modified mRNA can trigger a potent innate immune response in human fibroblasts, and that the reduction in immunogenicity achieved by incorporating these modified nucleotides may not be robust in the context of frequent transfection.

It has been demonstrated that with unmodified mRNA, suppressing the innate immune response of cells to exogenous RNA enables frequent transfection (Angel (2008); Angel & Yanik (2010), in which the use of B18R, a vaccinia-virus encoded decoy receptor for type I interferons, inhibits interferon signaling and enables frequent ivT mRNA transfection (Angel & Yanik (2010); Symons et al. (1995); Colamonici et al. (1995)). It appears from the results found herein that innate immune suppression may also be required for frequent transfection with modified mRNA, such as that containing pseudouridine and 5-methylcytidine.

Although it is exquisitely sensitive to exogenous RNA, at any given time a typical mammalian cell may contain more than 100,000 mRNA molecules, and many more rRNA and tRNA molecules, all of which evade detection by the cell's innate immune system. Several structural features have been identified that may contribute to the immunogenicity of viral RNA including the presence of a 5′ triphosphate and regions of secondary structure. However, these elements are not unique to viral RNA; tRNA contains a 5′ triphosphate and extensive secondary structure, and mRNA contains sequence elements that promote the formation of secondary structure in vitro, although the degree to which these structures actually form in vivo is less well understood. In addition, tRNA undergoes extensive post-transcriptional modification, including base modification of specific nucleotides. Interestingly, although mRNA is generally free of modified nucleotides, incorporating many of the modified nucleotides present in tRNA into ivT mRNA can reduce its immunogenicity (Kariko et al. (2004); Kariko et al. (2005)). It may be possible that the presence of modified nucleotides in tRNA may serve not only to stabilize its tertiary structure, but may also prevent tRNA from activating the innate immune system.

While the incorporation of many modified nucleotides into ivT mRNA are known to inhibit translation, Kariko et al. allege that incorporation of pseudouridine (Ψ) and 5-methylcytidine (5mC) does not inhibit translation, and that complete substitution of pseudouridine for uridine yields ivT mRNA with reduced immunogenicity that is translated into significantly more protein than unmodified mRNA both in vitro and in vivo (Kariko et al. (2008)). Recently, the authors explained the increased translation potential of pseudouridine-containing mRNA by showing that mRNA containing pseudouridine evades detection by PKR (Anderson et al. (2005)).

Results of experiments in which synthesis and transfection of cells with ivT mRNA containing no modifications, pseudouridine, 5-methylcytidine, or a combination of both modified nucleotides are shown herein. Although incorporation of modified nucleotides may reduce the immunogenicity of ivT mRNA, it is shown in Example 3 that this effect may be negligible in the context of frequent transfection. It is shown that a single transfection with modified mRNA triggers a potent immune response in primary human fibroblasts, and that innate immune suppression may be necessary both to achieve sustained, high-level expression of the encoded protein, and to rescue the cells from the massive cell death caused by frequent transfection with modified mRNA.

The interferon-stimulated gene IFIT1 is expressed at 10% of GAPDH after a single transfection with modified mRNA, which represents an approximately 100-fold upregulation compared to a vehicle-only control. High levels of the interferon-stimulated gene OAS1 (between 0.5 and 1% of GAPDH), were also detected while no expression of OAS1 was detected in the vehicle-only controls.

To test the ability of 5-methylcytidine incorporation to increase protein translation from pesudouridine-containing mRNA, Oct4-encoding RNA containing combinations of these modified nucleotides were synthesized. Fibroblasts were transfected with these modified mRNAs and the expression of Oct4 protein was measured by immunocytochemistry. In Example 4, it is shown that incorporation of pseudouridine increases protein translation from ivT mRNA, in agreement with previous results by Kariko et al. (2008).

However, as shown herein, the addition 5-methylcytidine to pseudouridine-containing mRNA decreases protein translation to a level comparable to or less than that of unmodified mRNA in fibroblasts. Additionally, it is shown that a previously published mRNA design, which incorporates the Cap 1 structure, yields increased protein translation compared to mRNA containing the standard Cap 0 cap in both modified as well as unmodified mRNA.

### Example 3

### Innate Immune Suppression Enables Frequent Transfection with Modified RNA

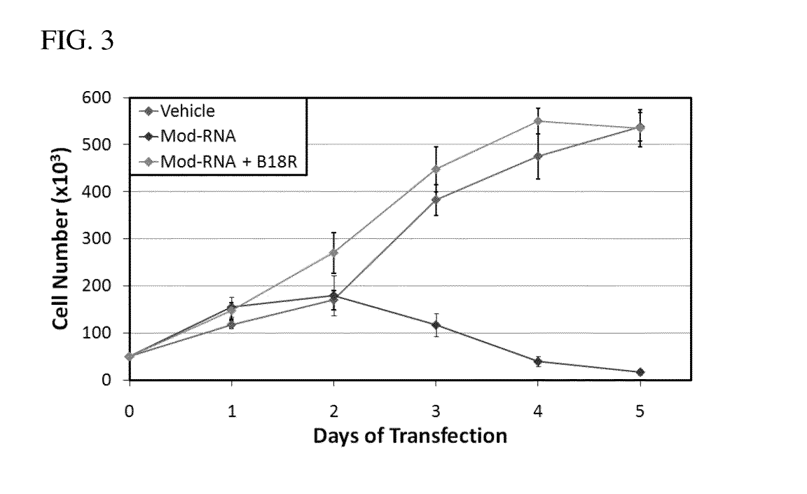

A mixture of ivT mRNA encoding Oct4, Sox2, Klf4, the tumor-promoting c-Myc T58A mutant (Hermann et al. (2005)), Lin28, and destabilized nuclear GFP was prepared as described by Warren, et al. MRC-5 human fetal lung fibroblasts were plated in 6-well plates at a density of 50,000 cells/well in DMEM+10% FBS, and 6 hours later the media was replaced with Nutristem+100 ng/mL bFGF or Nutristem+100 ng/mL bFGF+200 ng/mL B18R. Beginning the following day, cells were transfected every 24 hours for five days with 1.2 μg of modified mRNA as the authors described (FIG. 2). The culture medium (including supplements) was replaced daily. Transfected cells were morphologically indistinguishable from the vehicle-only control one day after the first transfection (day 1). However, beginning on day 2, an increase in the number of floating/dead cells was observed in the transfected wells, and by day 3 transfected wells exhibited the massive cell death that is characteristic of repeated transfection with unmodified mRNA. In contrast, in wells containing B18R, transfected cells proliferated rapidly, and remained at a density that was indistinguishable from the vehicle-only control throughout the course of the experiment (FIG. 3). A strong GFP signal was detected in transfected wells on day 1. By day 2 however, GFP expression was barely detectable, except in wells containing B18R, in which high levels of GFP were detected through day 5. A feeder layer was not included in this experiment, however similar results have been observed in experiments in which a feeder layer was included.

To examine the immunogenicity of modified mRNA, RNA was extracted from a sample of cells after a single transfection, and the expression of a panel of genes previously found to be upregulated following transfection with unmodified mRNA were measured (FIG. 1). Expression of IFIT1 and OAS1 was within a factor of two of the value previously reported by others (Warren, et al.), and expression of RIG1 was approximately 10-fold lower than the reported value. Expression of PKR was approximately 30-fold higher than the reported value, however expression of PKR in the vehicle-only control was approximately 10-fold higher than the reported value, likely reflecting differential expression of PKR in MRC-5 and BJ fibroblasts. Expression of IFNB1 was approximately 0.5% of GAPDH, which represents an approximately 7-fold upregulation relative to the vehicle-only control. The nearly identical upregulation of the two interferon-stimulated genes IFIT1 and OAS1 that were observed (and also reported by Warren et. al.), together with the lower expression of RIG-I that was observed lead to the conclusion that the modified mRNA used in the present experiments is not more immunogenic than that of Warren, et al.

In addition to the genes described above, also found was a >100-fold upregulation of IFIT2, IFIT3, OAS3, and OASL, and >50-fold upregulation of TLR3 following a single transfection with modified mRNA. In addition, high levels of expression of OAS1 and OAS2 were detected, two pattern recognition receptors for exogenous RNA that were not expressed in the vehicle-only control. In fact, a >5-fold upregulation of every gene in our panel was detected, indicating that a single transfection with modified mRNA had triggered a robust innate immune response in the fibroblasts. Additionally, many of these genes were further upregulated after a second transfection.

The expression of innate immune-related genes in cells transfected with modified mRNA was also measured, but cultured in media containing B18R (FIG. 1). It was found that expression of immune-related genes in our panel were reduced compared to cells not treated with the immunosuppressant, and that many of the genes that had been significantly upregulated in those cells (IFNB1, TLR3, EIF2AK2, STAT1, STAT2, IFIT5, OAS3, ISG20) were <2-fold upregulated compared to the vehicle-only control. In fact, the expression of genes in the panel was lower in cells transfected five times with modified mRNA and exposed to the immunosuppressant than in cells transfected only once with modified mRNA and not exposed to the immunosuppressant.

### Example 4

### RNA Containing Extensive Modifications is Translated Less Efficiently than Unmodified or Minimally-Modified RNA

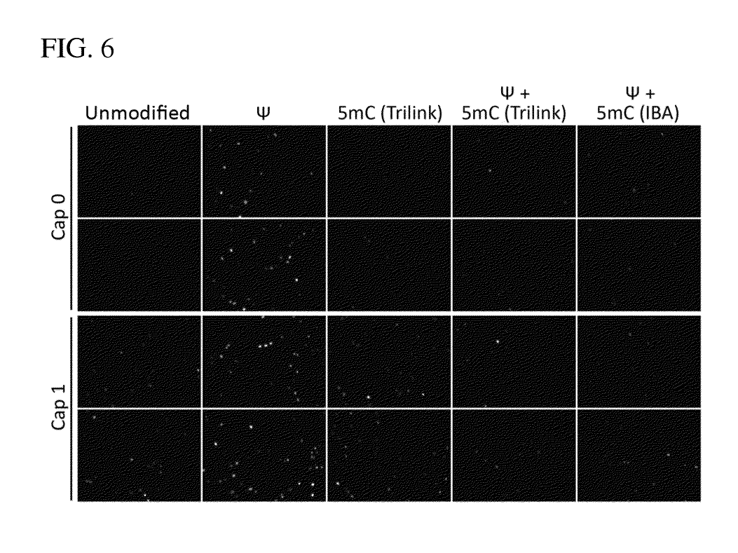

Having established that transfection with modified mRNA triggers a potent innate immune response in human fibroblasts, and that innate immune suppression rescues cells from the massive cell death caused by repeated transfection with modified mRNA, it was next sought to confirm whether the incorporation of 5-methylcytidine into pseudouridine-containing ivT mRNA enhances translation of the encoded protein as reported. To test this, capped, tailed mRNA encoding Oct4 and substituted Ψ-triphosphate, 5mC-triphosphate or both Ψ-triphosphate and 5mC-triphosphate for UTP and CTP in the in vitro-transcription reaction were synthesized. A previously published protocol was followed (Angel & Yanik (2010)) to generate RNA containing the Cap 1 structure, which has recently been shown to reduce the immunogenicity of RNA by inhibiting restriction by members of the IFIT family of pathogen recognition receptors (Daffis et al. (2010)). mRNA containing the Cap 0 structure was also synthesized, which more closely resembles the synthetic cap structure used by Warren, et al. Fibroblasts were plated in E-well plates at a density of 1×10⁵ cells/well. Several hours later, the media was replaced with Nutristem+100 ng/mL bFGF as before. The following day, the fibroblasts were transfected with 0.5 ug/well of the Oct4-encoding mRNA. The culture medium was replaced 4 hours after transfection, and the plates were fixed and stained for Oct4 protein 12 hours after transfection (FIG. 6).

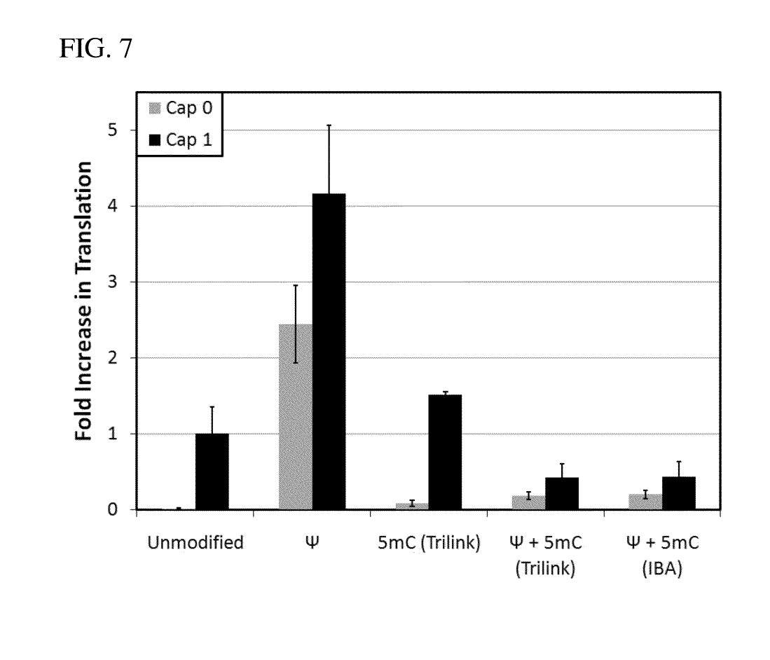

mRNA based on the design that has been previously described (unmodified, Cap 1) yielded many cells with brightly staining nuclei (FIG. 6). Incorporating Ψ increased the amount of translated protein by approximately 4 fold, while incorporating 5mC showed a negligible increase (FIG. 7). These results agree with the results presented by Kariko, et al. that Ψ increases protein translation from ivT mRNA, and that the effect of 5mC-incorporation is much more modest. Incorporating both Ψ and 5mC decreased the amount of protein translation relative to unmodified mRNA by roughly 2 fold. Nearly identical results were obtained from independent batches of mRNA encoding GFP and mCherry, and using 5-methylcytidine-triphosphate obtained from two different vendors. Similar results were also obtained using mRNA synthesized with the Cap 0 structure, although with every nucleotide combination, protein translation was significantly reduced when compared to the corresponding Cap 1 mRNA.

### Example 5

### Development of Culture Media for Efficient Nucleic Acid Transfection and Dedifferentiation

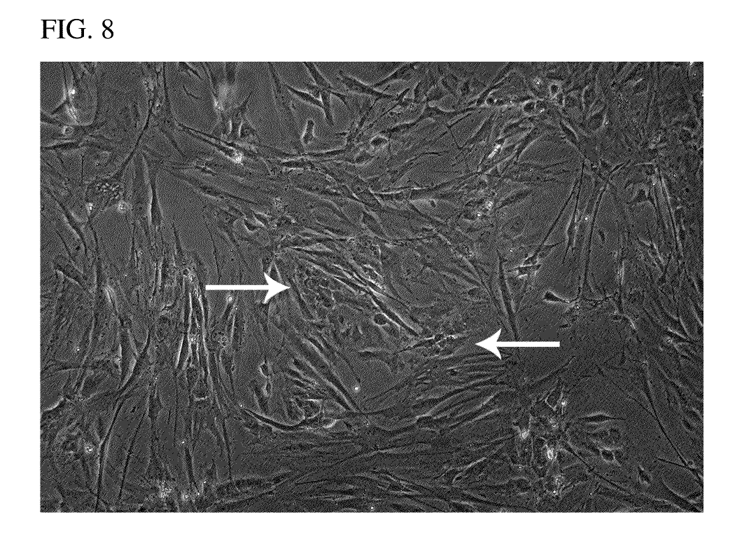

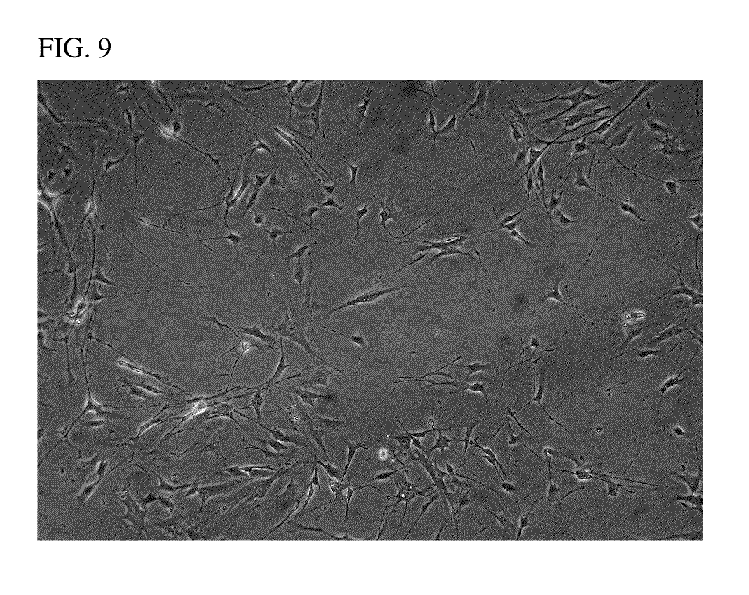

Twelve culture medium formulations (R1-R12) were developed that enable efficient dedifferentiation of cells. Culture media R1-R6 contain a surfactant (0.1% Pluronic F-68), which can increase the efficiency of transfection with nucleic acids. Although culture media for culturing stem cells have been previously described, such formulations contain components known to inhibit dedifferentiation (e.g., TGF-beta). FIG. 8 depicts the result of an experiment to dedifferentiate cells using a previously described medium containing TGF-beta. The white arrows show cells that begin to dedifferentiate, but then cease dedifferentiating due to the presence of TGF-beta. FIG. 9 depicts the results of an experiment to dedifferentiate cells using a medium that does not contain TGF-beta or a surfactant. The cells in this experiment did not undergo efficient dedifferentiation. FIG. 10 and FIG. 11 depict an experiment to dedifferentiate cells using the culture medium of the present invention (without TGF-beta or other inhibitors of dedifferentiation, but with a surfactant). The cells in this experiment were efficiently transfected, as evidenced by high-level expression of GFP (FIG. 10), and were efficiently dedifferentiated, as evidenced by clear morphological changes characteristic of dedifferentiation after only 9 days of transfection (FIG. 11). In all of the experiments described in this example, cells were dedifferentiated by repeated transfection with RNA encoding reprogramming proteins according to the present inventors' previously disclosed methods described in US Patent Appl. Pub. No. 2010/0273220.

### Medium R1:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF

### 0.1% Pluronic F-68

### Medium R2:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF

### 0.1% Pluronic F-68

0.5% human serum albumin

### Medium R3:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R

### 0.1% Pluronic F-68

### Medium R4:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R

### 0.1% Pluronic F-68

0.5% human serum albumin

### Medium R5:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R
200 nM dexamethasone

### 0.1% Pluronic F-68

### Medium R6:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R
200 nM dexamethasone

### 0.1% Pluronic F-68

0.5% human serum albumin

### Medium R7:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF

### Medium R8:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
0.5% human serum albumin

### Medium R9:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R

### Medium R10:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R
0.5% human serum albumin

### Medium R11:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R
200 nM dexamethasone

### Medium R12:

### DMEM/F12

### 2 mM L-alanyl-L-glutamine

5 ug/mL insulin
5 ug/mL transferrin
5 ng/mL selenous acid
4.5 ug/mL cholesterol
10 ug/mL cod liver oil fatty acids (methyl esters)
25 ug/mL polyoxyethylenesorbitan monooleate
2 ug/mL D-alpha-tocopherol acetate
1 ug/mL L-ascorbic acid 2-phosphate sesquimagnesium salt hydrate
20 ng/mL bFGF
200 ng/mL B18R
200 nM dexamethasone
0.5% human serum albumin

### REFERENCES

1. Angel M (2008) Extended Transient Transfection by Repeated Delivery of In Vitro-Transcribed RNA [Master's Thesis]. Cambridge, Mass.: Massachusetts Institute of Technology. 56 p.
2. Angel M, Yanik M F (2010) Innate immune suppression enables frequent transfection with RNA encoding reprogramming proteins. PLoS One 5: 1-7.
3. Takahashi K, Yamanaka S (2006) Induction of pluripotent stem cells from mouse embryonic and adult fibroblast cultures by defined factors. Cell 126: 663-676.
4. Okita K, Ichisaka T, Yamanaka S (2007) Generation of germline-competent induced pluripotent stem cells. Nature 448: 313-317.
5. Wernig M, Meissner A, Foreman R, Brambrink T, Ku M, et al. (2007) In vitro reprogramming of fibroblasts into a pluripotent ES-cell-like state. Nature 448: 318-324.
6. Takahashi K, Tanabe K, Ohnuki M, Narita M, Ichisaka T, et al. (2007) Induction of pluripotent stem cells from adult human fibroblasts by defined factors. Cell 131: 861-872.
7. Yu J, Vodyanik M A, Smuga-Otto K, Antosiewicz-Bourget J, Frane J L, et al. (2007) Induced pluripotent stem cell lines derived from human somatic cells. Science 318: 1917-1920.
8. Park I H, Zhao R, West J A, Yabuuchi A, Huo H, et al. (2008) Reprogramming of human somatic cells to pluripotency with defined factors. Nature 451: 141-146.
9. Lowry W E, Richter L, Yachechko R, Pyle A D, Tchieu J, et al. (2008) Generation of human induced pluripotent stem cells from dermal fibroblasts. Proc Natl Acad Sci USA 105: 2883-2888.
10. Yu J, Hu K, Smuga-Otto K, Tian S, Stewart R, et al. (2009) Human induced pluripotent stem cells free of vector and transgene sequences. Science 324: 797-801.
11. Jia F, Wilson K D, Sun N, Gupta D M, Huang M, et al. A nonviral minicircle vector for deriving human iPS cells. Nat Methods 7: 197-199.
12. Zhou H, Wu S, Joo J Y, Zhu S, Han D W, et al. (2009) Generation of induced pluripotent stem cells using recombinant proteins. Cell Stem Cell 4: 381-384.
13. Kim D, Kim C H, Moon J I, Chung Y G, Chang M Y, et al. (2009) Generation of human induced pluripotent stem cells by direct delivery of reprogramming proteins. Cell Stem Cell 4: 472-476.
14. Yakubov E, Rechavi G, Rozenblatt S, Givol D Reprogramming of human fibroblasts to pluripotent stem cells using mRNA of four transcription factors. Biochem Biophys Res Commun 394: 189-193.
15. Warren L, Manos P D, Ahfeldt T, Loh Y H, Li H, et al. (2010) Highly efficient reprogramming to pluripotency and directed differentiation of human cells with synthetic modified mRNA. Cell Stem Cell 7: 618-630.
16. Lin T, Ambasudhan R, Yuan X, Li W, Hilcove S, et al. (2009) A chemical platform for improved induction of human iPSCs. Nat Methods 6: 805-808.
17. Cooper O, Hargus G, Deleidi M, Blak A, Osborn T, et al. (2010) Differentiation of human ES and Parkinson's disease iPS cells into ventral midbrain dopaminergic neurons requires a high activity form of SHH, FGF8a and specific regionalization by retinoic acid. Mol Cell Neurosci 45: 258-266.
18. Kormann M S, Hasenpusch G, Aneja M K, Nica G, Flemmer A W, et al. (2011) Expression of therapeutic proteins after delivery of chemically modified mRNA in mice. Nat Biotechnol 29: 154-157.
19. Symons J A, Alcami A, Smith G L (1995) Vaccinia virus encodes a soluble type I interferon receptor of novel structure and broad species specificity. Cell 81: 551-560.
20. Colamonici O R, Domanski P, Sweitzer S M, Larner A, Buller R M (1995) Vaccinia virus B18R gene encodes a type I interferon-binding protein that blocks interferon alpha transmembrane signaling. J Biol Chem 270: 15974-15978.
21. Yoneyama M, Kikuchi M, Natsukawa T, Shinobu N, Imaizumi T, et al. (2004) The RNA helicase RIG-I has an essential function in double-stranded RNA-induced innate antiviral responses. Nat Immunol 5: 730-737.
22. Hornung V, Ellegast J, Kim S, Brzozka K, Jung A, et al. (2006) 5′-Triphosphate RNA is the ligand for RIG-I. Science 314: 994-997.
23. Saito T, Owen D M, Jiang F, Marcotrigiano J, Gale M, Jr. (2008) Innate immunity induced by composition-dependent RIG-I recognition of hepatitis C virus RNA. Nature 454: 523-527.
24. Takahasi K, Yoneyama M, Nishihori T, Hirai R, Kumeta H, et al. (2008) Nonself RNA-sensing mechanism of RIG-I helicase and activation of antiviral immune responses. Mol Cell 29: 428-440.
25. Yoneyama M, Fujita T (2008) Structural mechanism of RNA recognition by the RIG-I-like receptors. Immunity 29: 178-181.
26. Schmidt A, Schwerd T, Hamm W, Hellmuth J C, Cui S, et al. (2009) 5′-triphosphate RNA requires base-paired structures to activate antiviral signaling via RIG-I. Proc Natl Acad Sci USA 106: 12067-12072.
27. Schlee M, Roth A, Hornung V, Hagmann C A, Wimmenauer V, et al. (2009) Recognition of 5′ triphosphate by RIG-I helicase requires short blunt double-stranded RNA as contained in panhandle of negative-strand virus. Immunity 31: 25-34.
28. Kariko K, Ni H, Capodici J, Lamphier M, Weissman D (2004) mRNA is an endogenous ligand for Toll-like receptor 3. J Biol Chem 279: 12542-12550.
29. Kariko K, Buckstein M, Ni H, Weissman D (2005) Suppression of RNA recognition by Toll-like receptors: the impact of nucleoside modification and the evolutionary origin of RNA. Immunity 23: 165-175.
30. Kariko K, Muramatsu H, Welsh F A, Ludwig J, Kato H, et al. (2008) Incorporation of pseudouridine into mRNA yields superior nonimmunogenic vector with increased translational capacity and biological stability. Mol Ther 16: 1833-1840.
31. Anderson B R, Muramatsu H, Nallagatla S R, Bevilacqua P C, Sansing L H, et al. (2010) Incorporation of pseudouridine into mRNA enhances translation by diminishing PKR activation. Nucleic Acids Res.
32. Hemann M T, Bric A, Teruya-Feldstein J, Herbst A, Nilsson J A, et al. (2005) Evasion of the p53 tumour surveillance network by tumour-derived MYC mutants. Nature 436: 807-811.
33. Daffis S, Szretter K J, Schriewer J, Li J, Youn S, et al. (2010) 2′-O methylation of the viral mRNA cap evades host restriction by IFIT family members. Nature 468: 452-456.

What is claimed is:

1. A medium for transfecting a target cell with a ribonucleic acid molecule, the medium comprising DMEM/F 12, L-alanyl-L-glutamine, insulin, transferrin, selenous acid, cholesterol, cod liver oil fatty acids (methyl esters), polyoxyethylenesorbitan monooleate, D-alpha-tocopherol acetate, L-ascorbic acid 2-phosphate sesquimagnesium salt hydrate, and bFGF, wherein the medium is substantially free of TGF-beta.

2. The medium of claim 1 further comprising human serum albumin.

3. The medium of claim 1 further comprising a surfactant.

4. The medium of claim 3 wherein the surfactant is a non-ionic surfactant.

5. The medium of claim 1 further comprising an immunosuppressant.

6. The medium of claim 5 wherein the immunosuppressant is B18R.

7. The medium of claim 5 wherein the immunosuppressant is dexamethasone.

8. The medium of claim 1, wherein the medium supports growth of a somatic cell, growth of a stem cell, and dedifferentiation of a cell transfected with a ribonucleic acid molecule.

9. A method for transfecting a target cell with a ribonucleic acid molecule, the method comprising:
suppressing the innate immune response in the target cell; and
introducing the ribonucleic acid molecule into the target cell, wherein the target cell is cultured in a medium according to claim 1.

10. The method of claim 9, wherein the introduction of the ribonucleic acid molecule produces a phenotypic change in the target cell.

11. The method of claim 10, wherein the phenotypic change in the target cell is differentiation, transdifferentiation, or dedifferentiation.

12. The method of claim 9, wherein the target cell is a somatic cell.

13. A cell produced by the method of claim 9.
